(19) United States
(12) Patent Application Publication
(10) Pub. No.: US 2011/0054339 A1
(43) Pub. Date: Mar. 3, 2011

(54) METHOD FOR DETECTING RESPIRATORY CYCLES IN A STETHOSCOPE SIGNAL

(76) Inventors: Raymond Gass, Bolsenheim (FR); Sandra Reichert, Bischheim (FR)

(21) Appl. No.: 12/808,856

(22) PCT Filed: Dec. 18, 2008
(86) PCT No.: PCT/EP2008/010861

§ 371 Date: Nov. 15, 2010

(30) Foreign Application Priority Data
Dec. 18, 2007 (FR) 0759924

(51) Int. Cl. A61B 5/08

(52) U.S. Cl. 600/529

(57) ABSTRACT

In order to distinguish between a breathing phase and a non-breathing phase, it comprises steps consisting, for each stethoscope signal sample, of: —filtering (71) the stethoscope signal in order to eliminate the stethoscope signal's low frequencies, with the cutoff frequency preferentially being 500 Hz. —calculating (72) an energy value Eh for each sample of the filtered signal, —calculating (73) the mean energy Eh_moy of the filtered signal, —then making a decision (74), Breathing or Non-Breathing, based on the value of the difference Eh−Eh_moy for that sample.

The invention pertains to a method for detecting respiratory cycles in a stethoscope signal. In medicine, it is conventional to practice pulmonary auscultation using a stethoscope in order to obtain information on the physiology and pathologies of a patient's lungs and air passages. A physician seeks out particular sounds called markers, particularly sounds known as sibilant rales, crepitant rales, etc., in order to diagnose pathologies such as asthma or chronic obstructive pulmonary disease. Although conventional auscultation using a stethoscope is subjective and difficult to share, an electronic respiratory sound capture-and-analysis system should make it possible to assist the physician in performing an objective, timely diagnosis, thanks to its greater sensitivity and superior results reproducibility.

Creating such a system presents a problem in detecting respiratory cycles; more precisely, it is necessary to detect an interval of time corresponding to an inhalation phase, and another interval of time corresponding to an exhalation phase, said two intervals being separated by an non-breathing (apnea) interval. The inhalation and exhalation phases are both further subdivided into three parts: protophase (first third of the phase), mesophase (middle third of the phase, and telephase (last third of the phase). Automatic respiratory cycle detection is particularly useful for determining the number and position of the crepitant rales with respect to the respiratory cycle, and for monitoring sleep apnea.

The respiratory sounds may be detected by means of a sound sensor comprising a membrane (such as a stethoscope) and a microphone, said sensor being placed on the patient's mouth, or trachea, or lungs. The respiratory sounds detected in this manner shall hereafter be known as stethoscope sounds or the stethoscope signal. A distinction shall be made between pulmonary sounds, which are detected at the lungs, and tracheal sounds, which are detected at the trachea.

Stethoscope sounds are characterized by a broad spectrum with a mean frequency that depends on the sound detection point. The frequency of pulmonary sounds is generally assumed to fall within the 50-2500 Hz band, and that of tracheal sounds may reach as high as 4000 Hz. It is therefore possible to use a sampling frequency of 8 KHz. It is assumed that tracheal sounds have a spectrum of 60-600 Hz for inhalation and 60-700 Hz for exhalation.

Many noises overlap with the markers in which the physician is interested. In particular, the heart creates noise: The spectrum of cardiac sounds is from 20 to 100 Hz for basic signals, but it also includes higher frequencies (500 Hz and above) for sounds known as whistles. At the trachea, the normal respiratory sound is affected by a noise that contains high-frequency components, which are audible during both the inhalation phase and the exhalation phase. At the thorax, a normal respiratory sound is affected by a quiet noise during inspiration, and a very audible noise during the exhalation phase.

The respiratory signals are not stationary, because the volume of the lungs is constantly changing, and they vary based on the patient's age, body mass, and health condition. All of these factors make it difficult to automatically detect respiratory cycles. Various respiratory cycle detection systems have been tested, and most of these systems simultaneously make use of:
tracheal sounds, to determine the inhalation phase and the exhalation phase,
several pulmonary sounds,
and measuring the volume of air exhaled.

These known systems are experimental systems, which are too complex to be in common use among physicians, because they simultaneously use multiple sound sensors and a spirometer. Moreover, they may not be used by a patient alone for the purposes of home telemedicine. Furthermore, the respiratory cycle detection performed by these known systems is often disrupted by noise affecting the captured signals.

The document DE 10.2006.017.279 A1 describes a method for detecting respiratory cycles in a stethoscope signal, in order to distinguish between a breathing phase and a non-breathing phase, comprising the steps consisting, for each stethoscope signal sample, of:
calculating an energy value for each sample of the filtered signal, based on the values of a sequence of samples of the filtered signal, such as for a period of 200 ms,
calculating a reference value, which is the mean energy of the filtered signal, in a sliding window lasting several tens of seconds,
then making a decision, Breathing or Non-Breathing, based on the value of the difference between the energy calculated for that sample and the reference value.

This method only makes it possible to distinguish between a breathing phase and a non-breathing phase. It does not distinguish between inhalation and exhalation. Its purpose is to study sleep apnea. It is sufficient for detecting apnea. It could conceivably be used to conduct a first step of detecting breathing phases, before distinguishing between inhalation and exhalation. However, it has been observed that this first step of detecting breathing phases is insufficiently reliable at achieving reliable discrimination between inhalation and exhalation.

The purpose of the invention is to disclose a method and a system for automatically detecting respiratory cycles, achieving more reliable and robust detection with respect to spurious signals such as heart noises, noises from the sensor rubbing against the skin or clothes, ambient noises, the doctor's voice, etc.

The object of the invention is a method for detecting respiratory cycles in a stethoscope signal, in order to distinguish between a breathing phase and a non-breathing phase, comprising the steps consisting, for each stethoscope signal sample, of:
calculating an energy value Eh for each stethoscope signal sample, based on the values of a sequence of that signal's samples,
calculating the mean energy Eh_moy of that signal,
then making a decision, Breathing or Non-Breathing, based on the value of the difference Eh−Eh_moy for that sample;

characterized in that it consists of filtering the stethoscope signal using a high-pass filter, before calculating an energy value Eh for each sample of the stethoscope signal, and calculating the mean energy Eh_moy of that signal;

and in that the cutoff frequency is between 400 and 500 Hz.

Experiments show that the method characterized in this manner achieves more reliable breathing/non-breathing discrimination, because it eliminates disruptive noises (and part of the respiratory sounds) while allowing enough of the respiratory sounds through to enable reliable discrimination between breathing/non-breathing, and to enable, in a later step, reliable distinguishing between inhalation/exhalation.

In preferred embodiments, the inventive method comprises one or more of the following characteristics.

To calculate the mean energy Eh_moy of the filtered signal, it consists of considering all of the energy values Eh from the start of the filtered signal.

To make a smoothed decision for a sample Ei, it consists of:
considering a series of time windows Fj, with j varying from 1 to n, n being an even number. The time window Fj corresponds to the n consecutive samples
   Ei−n+j+1 . . . , Ei, . . . , Ei+j
For each window Fj, with j varying from 1 to n, counting within the window the number Rj of samples where the provisional decision is Breathing, and associating that number with each sample contained within the window Fj, particularly sample Ei,
adding up the values Rj for j=1 to n, which were respectively associated with the sample Ei for the time windows Fj, with j varying from 1 to n, in order to obtain a value

$$RT = \frac{( {\sum }_{j = 1}^{j = n} {R}_{j} )}{n} ,$$

then comparing the value RT to n/2 and subsequently concluding that the sample Ei belongs to a breathing phase if RT>n/2, or otherwise concluding that it belongs to a non-breathing phase.

According to one preferential embodiment, the method further comprises a step of smoothing the uncertain phases the duration of which is non-negligible with respect to the typical duration of a breathing phase or non-breathing phase, characterized in that, in order to smoothing a given uncertain phase, it consists of:
testing a first assumption whereby it is a breathing phase by checking the following conditions, said assumption being verified only if all of the following conditions are met:
   ∀j,k, Energy(aⱼ)≦Energy (rₖ)
         |Energy(rᵢ₊₂)−Energy(rᵢ₋₂)|<ε₂
         |Energy(rᵢ₊₁)−Energy(rᵢ₋₁)|<ε₁
         |Energy(rᵢ)−Energy(rᵢ₊₂)|<ε₂
         |Energy(rᵢ)−Energy(rᵢ₋₂)|<ε₂
   where aⱼ is a non-breathing phase and rₖ is a breathing phase,
   where ε₁, ε₂ are two fixed values,
   and where rᵢ is the uncertain phase, rᵢ₊₁ and rᵢ₊₂ are the two breathing phases that immediately follow it, and rᵢ₋₁ and rᵢ₋₂ are the two breathing phases that immediately precede it;
and if the first assumption is not verified, testing a second assumption, whereby it is a non-breathing phase, by checking the following conditions, said assumption being verified only if all of the following conditions are met:
   •j,k, Energy(aⱼ)≦Energy (rₖ)
   •j, |Energy(aᵢ)−Energy(aⱼ)|<ε₀
         |Energy(rᵢ₋₂)−Energy(rᵢ)|<ε₂
         |Energy(rᵢ₋₁)−Energy(rᵢ₊₁)|<ε₁
   where aⱼ is a non-breathing phase, aᵢ is a non-breathing phase, and rₖ is a breathing phase,
   where rᵢ is the uncertain phase, rᵢ₊₁ is the breathing phase that immediately follows it, rᵢ₋₁ and rᵢ₋₂ are the two breathing phases that immediately precede it,
   and where ε₀, ε₁, ε₂ are three fixed values.

According to one embodiment, in order to verify an assumption, the method further consists of measuring the duration of the uncertain phase and comparing it to a typical value corresponding to said assumption.

According to one embodiment, in order to verify an assumption, the method further consists of measuring the duration of the uncertain phase and comparing it to the mean value of the durations of other phases of the same type as the one defined by said assumption.

According to one embodiment, to distinguish between Inhalation/Exhalation within a breathing phase, it further consists of:
calculating the total energy of the samples of the even-numbered breathing phases, starting with the beginning of the signal,
calculating the total energy of the samples of the odd-numbered breathing phases, starting with the beginning of the signal,
comparing these two total energies, and deducing therefrom that the even-numbered breathing phases are inhalation phases if the total energy of the samples of the even-numbered breathing phases is greater than the total energy of the samples of the odd-numbered breathing phases, and vice versa.

According to another embodiment, to distinguish between Inhalation/Exhalation within a breathing phase, it further consists of:
calculating the mean of the durations of the even-numbered breathing phases,
calculating the mean of the durations of the odd-numbered breathing phases,
comparing these two means and deducing therefrom that the even-numbered breathing phases are exhalation phases if the mean of the durations of the even-numbered breathing phases is greater than the mean of the durations of the odd-numbered breathing phases, and vice versa.

The invention will be better understood, and other characteristics will become apparent, with the help of the description below and the figures accompanying it:

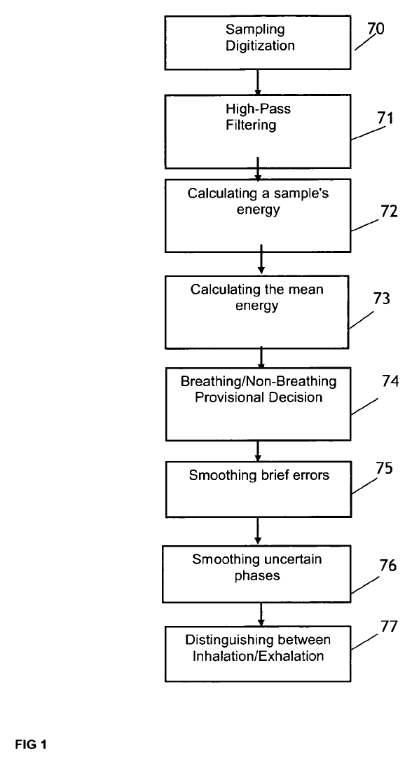

FIG. 1 depicts the steps of an example embodiment of the inventive method.

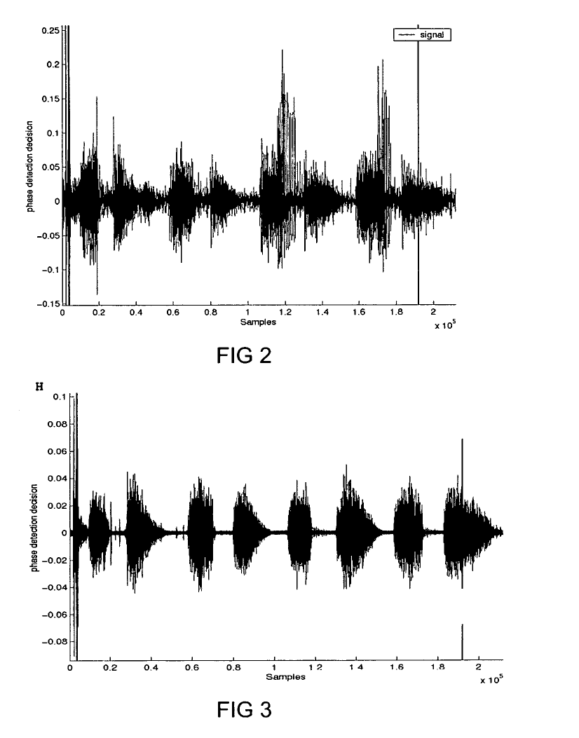

FIG. 2 depicts the graph of the value of a stethoscope signal detected at the lungs, during four respiratory cycles.

FIG. 3 depicts the graph of the value of this stethoscope signal, after high-pass filtering.

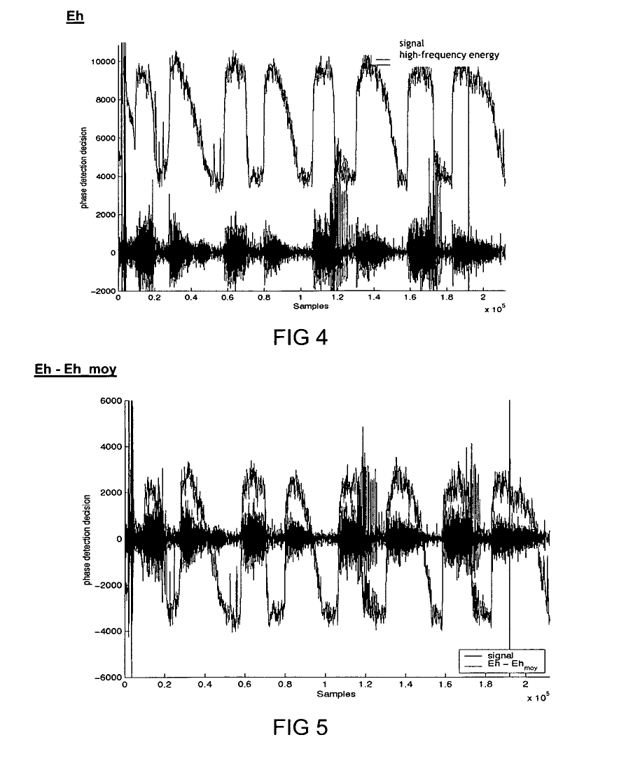

FIG. 4 depicts the graph of the energy of that same filtered stethoscope signal.

FIG. 5 depicts the graph of the difference between the energy of that same filtered stethoscope signal, and the mean energy of that same filtered stethoscope signal.

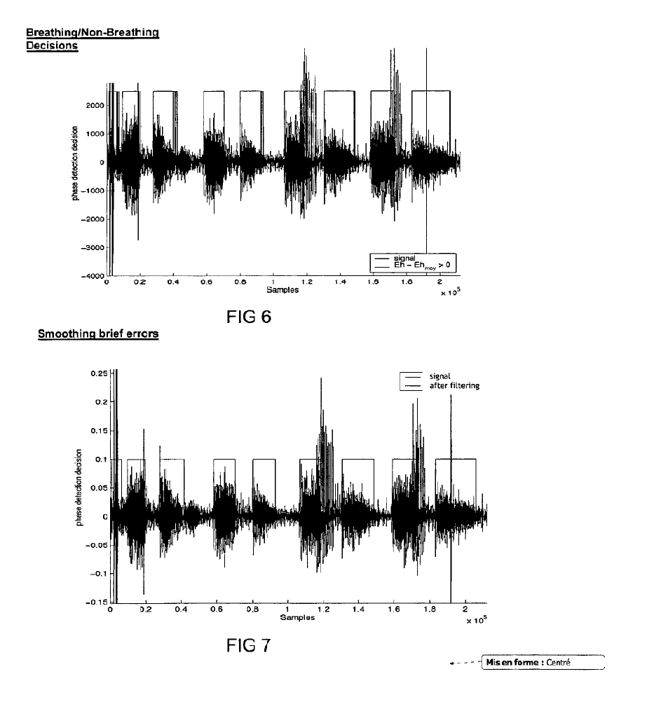

FIG. 6 depicts the graph of the provisional Breathing/Not-Breathing decisions, for the same filtered stethoscope signal.

FIG. 7 depicts the graph of the Breathing/Not-Breathing decisions, for the same filtered stethoscope signal, after smoothing the brief errors.

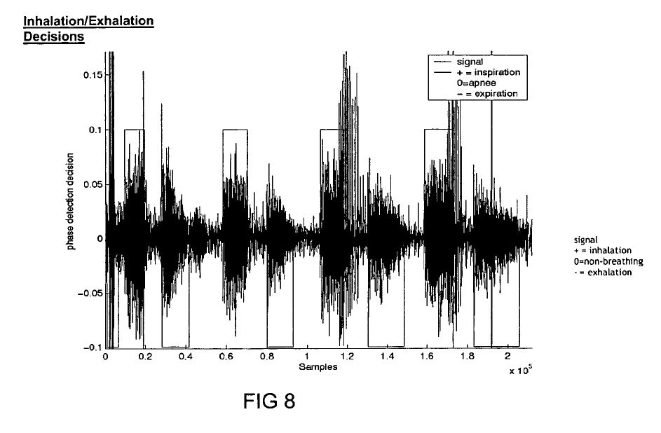

FIG. 8 depicts the graph of the Inhalation/Exhalation decisions during the breathing phases, for the same filtered stethoscope signal.

The phases of a respiratory cycle are detected in two successive steps, for each sound sample:

1) Distinguishing between a breathing phase (either inhalation or exhalation) and a non-breathing phase (just noise).

2) Distinguishing between inhalation and exhalation, for each breathing phase determined by the first distinguishing step.
The flowchart in FIG. 1 depicts the steps of an example embodiment of the inventive method.
Step 70: A respiratory sound is captured at the lungs, and then digitized at a frequency of 8 KHz. In one embodiment, the respiratory sound may be captured at the trachea.
Step 71: The signal is digitally filtered by a high-pass filter, having a cutoff frequency between 400 Hz and 500 Hz, and preferentially 500 Hz, to mitigate the noises that disrupt the detection of respiratory cycles, particularly noise due to the doctor's voice.
Step 72: Calculating an energy value Eh for each sample of the filtered signal, based on the values of a sequence of N samples of the filtered signal,
Step 73: Calculating the mean energy Eh_moy of the filtered stethoscope signal, over a time interval that preferentially begins at its start.
Step 74: Calculating the difference between the energy Eh calculated for a sample of the filtered stethoscope signal, and the mean energy Eh_moy of the filtered stethoscope signal. A provisional decision, Breathing or Non-Breathing, is made based on the value of this difference:

If (Eh−Eh_moy)>0, then it is a breathing phase.

Otherwise, it is a non-breathing phase.
Step 75: Smoothing the errors the duration of which is brief relative to the duration of a breathing or non-breathing phase. This makes it possible to eliminate incorrect decisions regarding an isolated sample or a few isolated samples.
Step 76: Smoothing the uncertain phases. An uncertain phase is an interval the duration of which is non-negligible when compared to the typical duration of a breathing phase or non-breathing phase, and which alternates between a low number of smoothed “Non-Breathing” decisions and a low number of smoothed “Breathing” decisions. This alternation is not smoothed by step 75, because it deals with too many samples. It creates one or more discontinuities in detecting a breathing phase or a non-breathing phase.
Step 77: Distinguishing between Inhalation/Exhalation during each breathing phase, using a method described further below.

The cutoff frequency of the high-pass filter 71 is between 400 Hz and 550 Hz, because it has been observed that with a low value, the rate of incorrect determinations increases rapidly. For example, this filtering may be achieved by a second-order Butterworth filter. To achieve a cutoff frequency of 500 Hz, the algorithm is as follows:

y[i]=b[0]*x[i]+b[1]*x[i−1]+b[2]*x[i−2]+a[1]*y[i−1]+a[2]*y[i−2]

where a(i) and b(i) are Butterworth filter coefficients.

In this embodiment:

b(0)=0.7571
b(1)=−1.5142
b(2)=0.7571
a(1)=−1.4542
a(2)=0.5741

Calculating 72 the energy Eh associated with each sample of the filtered stethoscope signal is using a conventional method. It is calculated for a given sample, taking into consideration a window containing the 240 samples that precede it. The calculation period is thus equal to the sampling period.

The energy E of a signal in the discrete domain is calculated using the formula:

$$E = {\sum }_{n} {x}^{2} ( i ) where x ( i ) is the value of the signal ' s nth sample .$$

We take into consideration a 240-sample window (i.e. 30 ms for a signal with a sampling frequency of 8 kHz).

$$Hence E = {\sum }_{n = 0}^{239} {x}^{2} ( i )$$

where x(i) is encoded using 16 bits, and these values vary between −2¹⁵ and 2¹⁵. The energy E therefore takes on values between 0 and 240*2³¹.

In order to achieve results independent of the size of the window in question, we will divide the obtained value of E by the number of samples in the window, N=240.

Additionally, in order to simplify the implementation, we make a switch to logarithmic plotting. This reduces the dynamics, but has no effect on the results achieved. Finally, the formula of the energy Eh for each sample is:

$$Eh = log ( \frac{{\sum }_{n = 0}^{N - 1} {x}^{2} ( i )}{N} ) with N = 240$$

The mean energy Eh_moy of the filtered stethoscope signal is calculated, in step 73, over an interval of time that preferentially begins at its start, in order to eliminate patient-dependent variations and to eliminate the effect of spurious noises. When the doctor begins auscultation, he applies the stethoscope's bell onto the patient's skin, and moves it. The movement produces a rubbing noise. At the same time, he speaks, such as to say “breathe in deeply”. Next, he focuses on what he hears, and waits for a period of time in one spot, then moves the stethoscope's bell again over the patient's skin.

Calculating the mean energy Eh_moy over a fixed period, no matter how long, does not guarantee that the calculation is not conducted over a bad period, when the bell is being moved. Calculating the mean from the beginning of the signal makes it possible to benefit from the fact that the total duration of the movement is much less than the total duration of non-movement. Thus, the mean value calculated is much closer to the ideal mean, which would only take into account periods when there is no spurious noise due to the bell moving, and to the doctor's voice.

FIG. 2 shows the graph of the value V of the stethoscope signal captured over four respiratory cycles (about 180,000 samples). Whenever a doctor asks the patient to inhale and exhale completely, each cycle generally lasts between 4 and 7 seconds. Each cycle includes: an inhalation phase, a non-breathing phase, an exhalation phase, and a second non-breathing phase. During a non-breathing phase, the lack of air movements means no sound is produced, but the sensor captures noise. This figure shows that the sound volume during the inhalation phase is much greater than during the exhalation phase. The noise volume during the two non-breathing phases is generally much lower than the respiratory sound volume during the exhalation phase, but it is not negligible compared to the respiratory sound volume. Furthermore, sometimes the noise is greater than the respiratory sound volume, particularly vocal noise when the doctor is speaking to the patient (second half of the graph).

FIG. 3 depicts the graph of the value Vh of the same stethoscope signal after high-pass filtering, with a cutoff frequency of 500 Hz. It has been observed that noise, in particular vocal noise, is much lower compared to the original signal shown in FIG. 2.

FIG. 4, in its upper portion, depicts the graph of the energy Eh of that same filtered stethoscope signal, which is depicted in the lower portion.

FIG. 5 depicts the graph of the difference (Eh−Eh_moy) between the energy Eh of the same filtered stethoscope signal, and the mean energy Eh_moy of that same filtered stethoscope signal. Based on this data, a provisional Breathing/Not-Breathing decision is made, by conducting the following test:

If (Eh−Eh_moy)>0, then it is a breathing phase.

Otherwise, it is a non-breathing phase.

Subtracting this sliding mean value Eh_moy as references makes it possible to constantly adapt the distinguishing conditions to variations in the noise level and to variations in the useful signal level, these levels being different, particularly from one patient to another.

FIG. 6 depicts the graph of the provisional Breathing/Not-Breathing decisions, for the same filtered stethoscope signal, over four respiratory cycles. The graph of this filtered signal is superimposed.

Smoothing Brief Errors (Step 75)

For each sample, a provisional decision is made, in step 74:
Either Breathing (abbreviated “r”) if (Eh−Emoy)>0
Or Non-Breathing (abbreviated “a”) if (Eh−Emoy)<or =0

Ideally, a series of decisions of the following form is obtained:

r r r r r r r r r r r r r r r r a a a a a a a a r r r r r r r r r r r r r a a a a a a a

However, incorrect decisions may occur, for one sample or several consecutive samples. To eliminate these incorrect decisions, the brief errors are smoothed. These decisions will hereafter be called “smoothed decisions”

A given sample Ei is considered, for which a provisional decision DPi has been made, and for which a smoothed decision DLi should be determined. The provisional decision DPi and the provisional decisions made for the n samples immediately preceding the given sample Ei will be used. Based on these n+1 provisional decisions, this smoothed decision DLi is determined by a calculation which is conducted some time after the calculation of the provisional decision DPi.

A sliding time window is considered, whose size corresponds to n samples, n being a fixed even number. The sampling period is called T. This time window is shifted by a sampling period T for each new sample, so that a series of windows F0, F1, F2, F3, . . . is obtained. The temporal shift between the provisional decision and the final decision, for a single sample, is equal to Tn or greater and is fixed. This makes it possible to have n+1 provisional decisions DPi−n, DPi−n+1 . . . DPi−1, Dpi, in order to make a smoothed decision DLi.

At a moment t0, a time window F0 corresponds to the given sample Ei and the n−1 samples that precede it:

Ei−n+1, . . . , Ei−1, Ei.

At the moment t1=t0+T, a new time window F1 corresponds to the samples:

Ei−n+2, . . . , Ei, Ei+1.

At the moment t2=t1+T, a new time window F3 corresponds to the samples:

Ei−n+3, . . . , Ei, Ei+1, Ei+2.

At the moment t3=t2+2T, a new time window F4 corresponds to the samples:

Ei−n+4 . . . Ei, Ei+1, Ei+2, Ei+3.

At the moment tj=t1+j.T, a new time window Fj corresponds to the samples:

Ei−n+j+1 . . . Ei+j.

At the moment tn=t1+n.T, a new time window Fn corresponds to the samples:

### Ei, . . . , Ei+n.

It should be noted that each sample is contained within a series of n windows shifted apart from one another. At the moment tn, it is possible to make a smoothed decision DLi for the sample Ei, as the provisional decisions made for the samples contained in all the windows containing that given sample Ej, i.e. windows F0 to Fn, are then known.

For each window Fj, within the window, the number of samples where the provisional decision is “Breathing” is counted. The number obtained, Rj, is between 0 and n inclusive. This number Rk is associated with each sample contained within the window Fj, particularly the sample Ei, because this number represents the likelihood of the Breathing decision in this window:

The value R0 is associated with all the samples in the window F0.
The value R1 is associated with all the samples in the window F1.
The value Rj is associated with all the samples in the window Fj.
The value Rn is associated with all the samples in the window Fn.

In order to determine the smoothed decision DLi for the sample Ei, the n windows Fj are considered, as are the corresponding values Rj, for j=1 to n. These values Rj are added up for j=1 to n, in order to obtain a value RT representative of the likelihood of the Breathing decision. Next, the following test is conducted:

$$If RT = \frac{( {\sum }_{j = 1}^{j = n} {R}_{j} )}{n} > n / 2$$

Then it is a breathing sample.

Otherwise, it is a non-breathing sample.

It is assumed that the samples at the start of the signal are not important to the analysis that will follow; the provisional decision is therefore arbitrarily set to Non-Breathing for the first n samples, at the start of the signal.

Example where n=8.

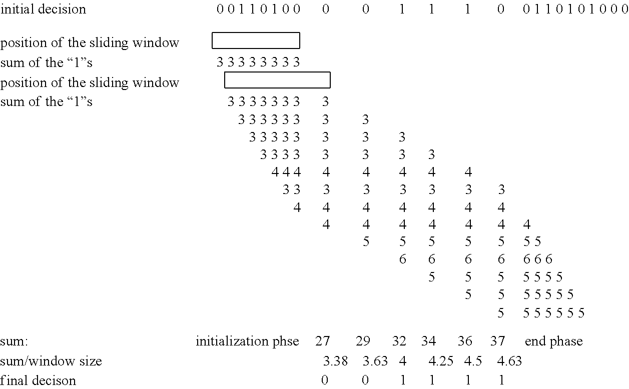

1 = decision: it's breathing
0 = decision: it's non-breathing
in the example, an 8-sample window is considered

FIG. 7 depicts the graph of the Breathing/Non-Breathing decisions, for the same filtered stethoscope signal, as in FIGS. 1-6, after smoothing the brief errors. In order to better display the impact of smoothing, FIG. 7 also depicts the results before and after smoothing.

Smoothing Uncertain Phases (Step 76)

As a reminder, an uncertain phase is an interval the duration of which is non-negligible when compared to the typical duration of a breathing phase or non-breathing phase, and which alternates between a low number of smoothed “Non-Breathing” decisions and a low number of smoothed “Breathing” decisions. This alternation, which is not smoothed by step 75, creates one or more discontinuities when detecting a breathing or a non-breathing phase.

Uncertain phases are a situation that we did not encounter during our experiments. However, we have nevertheless provided for the possibility that such a situation may occur.

In the pulmonary auscultatory signals that are to be analyzed, the respiratory cycles are “inhalation-non-breathing-exhalation-non-breathing”. A breathing phase should cause a continuous series of “r” decisions after the brief error smoothing step. A non-breathing phase should cause a continuous series of “a” decisions after the brief error smoothing step. A transition from “r” to “a” should make it possible to conclude that it is the end of a breathing phase and the start of a non-breathing phase. A transition from “a” to “r” should make it possible to conclude that it is the end of a non-breathing phase and the start of a breathing phase.

Ideally, the brief error smoothing step should therefore give as a result like this one:

. . . r . . . |_a_| . . . r . . . |_a_| . . . r . . . |_a_| . . . r . . . |

However, uncertain phases may appear. An uncertain phase will be denoted as “uncertain”. There are 4 possible situations:

Situation a: an uncertain phase appears between two breathing phases.
| . . . r . . . |_a_| . . . r . . . |uncertain| . . . r . . . |_a_| . . . r . . . |
Situation a1: an uncertain phase appears between two non-breathing phases.
| . . . r . . . |_a_| . . . r . . . |_a_|uncertain|_a_| . . . r . . . |
Situation b: an uncertain phase appears between a breathing phase and a non-breathing phase.
| . . . r . . . |_a_| . . . r . . . |uncertain|_a_| . . . r . . . |_a_| . . . r . . . |
Situation b1: an uncertain phase appears between a non-breathing phase and a breathing phase.
| . . . r . . . |_a_| . . . r . . . |_a_|uncertain| . . . r . . . |_a_| . . . r . . . |

Whatever the situation, the same method is used to determine whether it is a breathing phase or a non-breathing phase. To do so, the following two hypotheses are tested. If the first assumption is incorrect, the second one is checked.

Assumption 1: This is a breathing phase rᵢ. This assumption is accurate if the following tests both give a positive result:
Phase energy test: The cycles are known to follow the “inhalation-non-breathing-exhalation-non-breathing” model (alternating between inhalation and exhalation). The signal's energy, calculated over the duration of a non-breathing phase, is less than the signal's energy calculated over a breathing phase. Additionally, the signal's energy calculated over an inhalation phase is greater than the signal's energy calculated over an exhalation phase. The signal's energy is calculated during each phase, and the energies of the even-numbered breathing phases and odd-numbered breathing phases are compared. If one of the following conditions is not met, then assumption 1 is false:
•j,k, Energy(aⱼ)≦Energy (rₖ)
   |Energy(rᵢ₊₂)−Energy(rᵢ₋₂)|ε₂
   |Energy(rᵢ₊₁)−Energy(rᵢ₋₁)|<ε₁
   |Energy(rᵢ)−Energy(rᵢ₊₂)|<ε₂
   |Energy(rᵢ)−Energy(rᵢ₋₂)|<ε₂
   where aⱼ is a non-breathing phase and rₖ is a breathing phase,
   where ε₁, ε₂ are two fixed values,
   and where rᵢ is the uncertain phase, rᵢ₊₁ and rᵢ₊₂ are the two breathing phases that immediately follow it, and rᵢ₋₁ and rᵢ₋₂ are the two breathing phases that immediately precede it;
Phase duration test: The typical duration of a breathing phase is between 1.5 and 3.5 s. The typical duration of a non-breathing phase is between 0.5 and 2.5 s. The durations of the various phases are measured. If a major inconsistency is detected between the duration of the uncertain phase and predetermined typical durations (for example, a breathing phase duration equal to 7s), this means that the assumption is incorrect.

In ambiguous cases, it is also possible to calculate the mean duration of the inhalation phases, the mean duration of the exhalation phases, and the mean duration of the non-breathing phases, for the signal being considered, i.e. for a particular patient; and to compare the duration of the uncertain phase with the determined averages.

Assumption 2: The uncertain phase is a non-breathing phase aⱼ. This assumption is accurate if the following tests both give a positive result:
Phase energy test: The signal's energy during each phase is calculated, and the energy of an even-numbered breathing phase is compared to the energy of an odd-numbered breathing phase. If one of the following conditions is not met, then assumption 2 is false:
•j,k, Energy(aⱼ)≦Energy (rₖ)
•j, |Energy(aᵢ)−Energy(aⱼ)|<ε₀
   |Energy(rᵢ₋₂)−Energy(rᵢ)|<ε₂
   |Energy(rᵢ₋₁)−Energy(rᵢ₊₁)|<ε₁
where aⱼ is a non-breathing phase, aᵢ is a non-breathing phase, and rₖ is a breathing phase,
where rᵢ is the uncertain phase, rᵢ₊₁ is the breathing phase that immediately follows it, rᵢ₋₁ and rᵢ₋₂ are the two breathing phases that immediately precede it,
and where ε₀, ε₁, ε₂ are three fixed values.
Phase duration test: The duration of the various phases is measured. If a major inconsistency is detected compared with predetermined typical durations (for example, a non-breathing phase duration equal to 7s), this means that the assumption is incorrect.
In ambiguous cases, it is also possible to calculate the mean duration of the inhalation phases, the mean duration of the exhalation phases, and the mean duration of the non-breathing phases, for the signal being considered, i.e. for a particular patient; and to compare the duration of the uncertain phase with the determined averages.

### Distinguishing Between an Inhalation Phase and an Exhalation Phase (Step 77)

When distinguishing between Breathing/Non-Breathing, the breathing phases were determined. This makes it possible to eliminate the samples of the signal corresponding to the non-breathing phases. The remaining signal samples correspond only to inhalation phases and exhalation phases. The remaining series of samples theoretically alternates between an inhalation phase and an exhalation phase. Two situations are possible:
Either all the even-numbered breathing phases correspond to inhalation, in which case all the odd-numbered breathing phases correspond to exhalation.
Or all the even-numbered breathing phases correspond to exhalation, in which case all the odd-numbered breathing phases correspond to inhalation.

It is known that the signal's energy calculated over the duration of an inhalation phase is generally greater than the energy of the signal calculated over the duration of an exhalation phase.

According to one preferential embodiment, the method for distinguishing between Inhalation/Exhalation consists of:
calculating the total energy of the samples of the even-numbered breathing phases, starting with the beginning of the signal,
calculating the total energy of the samples of the odd-numbered breathing phases, starting with the beginning of the signal,
comparing these two total energies, and deducing therefrom that the even-numbered breathing phases are inhalation phases if the total energy of the samples of the even-numbered breathing phases is greater than the total energy of the samples of the odd-numbered breathing phases, and vice versa.

FIG. 8 depicts the graph of the Inhalation/Exhalation decisions during the breathing phases, for the same filtered stethoscope signal. Each inhalation phase is depicted in the upper part of the graph, superimposed on the original signal. Each exhalation phase is depicted in the lower part of the graph.

A second method for distinguishing between Inhalation/Exhalation may consist of:
calculating the mean of the durations of the even-numbered breathing phases,
calculating the mean of the durations of the odd-numbered breathing phases,
comparing these two means and deducing therefrom that the even-numbered breathing phases are exhalation phases if the mean of the durations of the even-numbered breathing phases is greater than the mean of the durations of the odd-numbered breathing phases, and vice versa.

According to an preferred embodiment of the inventive method, the first method for distinguishing between Inhalation/Exhalation is used for distinguishing between Inhalation and Exhalation, then the second method is used to check the accuracy of the distinguishing action performed by the first method.

1) A method for detecting respiratory cycles in a stethoscope signal, in order to distinguish between a breathing phase and a non-breathing phase, comprising the steps consisting, for each stethoscope signal sample, of:
calculating (72) an energy value Eh for each stethoscope signal sample, based on the values of a sequence of that signal's samples,
calculating (73) the mean energy Eh_moy of that signal,
then making a decision (74), Breathing or Non-Breathing, based on the value of the difference Eh−Eh_moy for that sample;
characterized in that it consists of filtering (71) the stethoscope signal using a high-pass filter, before calculating (72) an energy value Eh for each sample of the stethoscope signal, and calculating (73) the mean energy Eh_moy of that signal;
and in that the cutoff frequency is between 400 and 500 Hz.

2) A method according to claim 1, characterized in that, in order to calculate (73) the mean energy Eh_moy of the filtered signal, it consists of considering all the energy values Eh starting from the beginning of the filtered signal.

3. A method according to claim 1, characterized in that it further comprises a step (75) of smoothing brief errors, and in that in order to determine a smoothed decision for a sample Ei, it consists of:
considering a series of time windows Fj, with j varying from 1 to n, n being an even number, the time window Fj corresponding to the n consecutive samples
Ei−n+j+1 . . . , Ei, . . . , Ei+j
For each window Fj, with j varying from 1 to n, counting within the window the number Rj of samples where the provisional decision is Breathing, and associating that number with each sample contained within the window Fj, particularly sample Ei,
adding up the values Rj for j=1 to n, which were respectively associated with the sample Ei for the time windows Fj, with j varying from 1 to n, in order to obtain a value
$$RT = \frac{( {\sum }_{j = 1}^{j = n} {R}_{j} )}{n} ,$$
then comparing the value RT to n/2 and subsequently concluding that the sample Ei belongs to a breathing phase if RT>n/2, and otherwise concluding that it belongs to a non-breathing phase.

4) A method according to claim 1, characterized in that it further comprises a step (76) of smoothing the uncertain phases which have a non-negligible duration compared to the typical duration of a breathing phase or non-breathing phase, characterized in that, in order to smooth a given uncertain phase, it consists of:
testing a first assumption whereby it is a breathing phase by checking the following conditions, said assumption being verified only if all of the following conditions are met:
•j,k, Energy(aⱼ)≦Energy (rₖ)

|Energy(rᵢ₊₂)−Energy(rᵢ₋₂)|<ε₂

|Energy(rᵢ₊₁)−Energy(rᵢ₋₁)|<ε₁

|Energy(rᵢ)−Energy(rᵢ₊₂)|<ε₂

|Energy(rᵢ)−Energy(rᵢ₋₂)|<ε₂
where aj is a non-breathing phase and rk is a breathing phase,
where ε₁, ε₂ are two fixed values,
and where rᵢ is the uncertain phase, rᵢ₊₁ and rᵢ₊₂ are the two breathing phases that immediately follow it, rᵢ₋₁ and rᵢ₋₂ are the two breathing phases that immediately precede it;
and if the first assumption is not verified, testing a second assumption, whereby it is a non-breathing phase, by checking the following conditions, said assumption being verified only if all of the following conditions are met:
•j,k, Energy(aⱼ)≦Energy (rₖ)
•j, |Energy(ai)−Energy(aⱼ)|<ε₀

|Energy(rᵢ₋₂)−Energy(rᵢ)|<ε₂

|Energy(rᵢ₋₁)−Energy(rᵢ₊₁)|<ε₁
where aⱼ is a non-breathing phase, aᵢ is a non-breathing phase, and rₖ is a breathing phase,
where rᵢ is the uncertain phase, rᵢ₊₁ is the breathing phase that immediately follows it, rᵢ₋₁ and rᵢ₋₂ are the two breathing phases that immediately precede it, and where ε₀, ε₁, ε₂ are three fixed values.

5) A method according to claim 4, characterized in that, in order to verify an assumption, it further consists of measuring the duration of the uncertain phase and comparing it to a typical value corresponding to said assumption.

6) A method according to claim 4, characterized in that, in order to verify an assumption, it further consists of measuring the uncertain phase and comparing it to the mean value of the durations of other phases of the same type as the one defined by said assumption.

7) A method according to claim 1, characterized in that in order to distinguish between Inhalation and Expiration within a breathing phase, it further consists of:
calculating the total energy of the samples of the even-numbered breathing phases, starting with the beginning of the signal,
calculating the total energy of the samples of the odd-numbered breathing phases, starting with the beginning of the signal,
comparing these two total energies, and deducing therefrom that the even-numbered breathing phases are inhalation phases if the total energy of the samples of the even-numbered breathing phases is greater than the total energy of the samples of the odd-numbered breathing phases, and vice versa.

8) A method according to claim 1, characterized in that in order to distinguish between Inhalation and Expiration within a breathing phase, it further consists of:
calculating the mean of the durations of the even-numbered breathing phases,
calculating the mean of the durations of the odd-numbered breathing phases,
comparing these two means and deducing therefrom that the even-numbered breathing phases are exhalation phases if the mean of the durations of the even-numbered breathing phases is greater than the mean of the durations of the odd-numbered breathing phases, and vice versa.

9) A method according to claim 1, characterized in that in order to filter (71) the stethoscope signal, the cutoff frequency is equal to 500 Hz.

10) A method according to claim 1 implemented by a programmable device comprising storage means in which a program is saved, said program comprising instructions which, when they are executed, carry out the steps of the method.

11) A method according to claim 1 implemented by a storage means in which a program is saved, said program comprising instructions which, when they are executed in a programmable device, carry out the steps of the method.

12) A method according to claim 1 implemented by a device comprising means suitable for executing steps of the method.
